(19) United States
(12) Patent Application Publication
(10) Pub. No.: US 2013/0018351 A1
(43) Pub. Date: Jan. 17, 2013

(54) Package associating disposable articles structured for reduced chafing

(71) Applicant: Fred Naval Desai, Fairfield, OH (US)

(72) Inventor: Fred Naval Desai, Fairfield, OH (US)

(73) Assignee: The Procter & Gamble Company, Cincinnati, OH (US)

(21) Appl. No.: 13/182,950

(22) Filed: Jul. 14, 2011

(51) Int. Cl. A61F 13/49; B26D 1/12; A61F 13/84

(52) U.S. Cl. 604/385.29; 604/385.01; 493/370

(57) ABSTRACT

A package associating disposable absorbent articles structured for reduced chafing is disclosed. The package may associate at least two sets of articles, the articles in the respective sets having respectively differing structures, so that a user has the differing articles available to apply to a wearer. The respective structures may be varied to effect differing contact locations and pressures about the waist and legs of the wearer. The time over which the wearer continuously wears articles of unvarying fit may thereby be reduced, reducing the likelihood or extent of continuous contact and pressure by articles at concentrated locations of the wearer's skin and body. Likelihood of irritation and chafing may thereby be reduced. An associated method for reducing skin irritation or chafing is disclosed. A method for manufacturing such a package by manufacturing differing structures on a single manufacturing line is disclosed.

### FIELD OF THE INVENTION

The present invention relates generally to the field of disposable wearable absorbent articles (e.g., disposable diapers), and more particularly, packages of sets of such articles.

### BACKGROUND OF THE INVENTION

Currently many parents and caregivers of babies purchase and apply disposable wearable absorbent articles to their babies for containment and absorption of bodily exudates during their babies' early years. Older children experiencing childhood enuresis, and adults experiencing incontinence, may wear disposable wearable absorbent articles as well.

In the past, such articles as manufactured had relatively less elastic stretch/contraction capability about areas such as the waist and leg openings. As a result, such articles could have a relatively looser fit about the wearer's waist and legs resulted in gaps between the article chassis and the wearer's skin at these areas, rendering the articles more susceptible to leakage of bodily exudates. On the other hand, the relatively less, and less constant, skin contact by the article about the waist and leg openings meant less likelihood of skin marking, initiation and chafing. Additionally, the gaps allowed for greater movement of air into and out of the spaces between the article and the wearer's body, reducing the likelihood of skin overhydration, which can promote undesirable conditions such as diaper rash.

For purposes of improvement of appearance, fit, retention of the article on the wearer, and exudate containment, many currently marketed disposable wearable absorbent articles are designed with features such as elasticized barrier cuffs and leg bands, and elasticized waist bands or elasticized waist regions. Such features help ensure a snug fit about the edges of the article containment structure, i.e., the crotch areas, leg openings and waist opening, and substantially reduce the possibility that exudates can leak from or escape the article structure and soil surrounding clothing, bedding, furniture, etc.

Such elasticized features, however, cause closer and more frequent and/or sustained skin contact and pressure on the skin, and may promote skin marking, irritation and chafing as a result of continuous and repeated wear, especially when an article is loaded with urine. Elasticized leg bands and an elasticized waist band or waist region may be in nearly constant contact with the wearer's skin in these areas during the period of wear of the article. The structure of the article may apply pressure to the skin about the leg hoop area, the waist hoop area, and any other area of the wearer's body where an elastic or elasticized member creates a zone of hoop-wise tension, especially when the article is loaded by the weight, and or stretched by, absorbed urine. When a disposable wearable absorbent article is removed, it is often replaced with another. As a result, a wearer may have such leg bands and a waist band or waist region in contact with her skin over extended periods of time, e.g., many hours of each day. The wearer's movements may cause the leg bands and waist band/waist region to shift about and rub against the wearer's skin. As a result of such extended wear and rubbing, the wearer may suffer irritation and/or chafing of her skin in the areas beneath the leg bands and waist band/waist region. Such irritation and/or chafing may be exacerbated if the wearer's skin beneath the article is over-hydrated, which sometimes can occur when the article does not adequately breath or cannot otherwise adequately vent humid air from within its structure, and/or when a wet article is not quickly changed.

In view of the concerns identified above, it would be advantageous if features could be provided to reduce the possibility for skin marking, irritation and/or chafing, while still providing the fit and containment advantages of features of current disposable wearable absorbent articles.

### BRIEF DESCRIPTION OF THE DRAWINGS

FIG. 1 is a perspective view generally of the body-facing surfaces of a disposable wearable absorbent article in the form of a diaper shown in a relaxed condition before application to a wearer;

FIG. 2 is a perspective view generally of the outer surfaces of a disposable wearable absorbent article in the form of a diaper shown in a relaxed condition before application to a wearer;

FIG. 3 is a plan view of a disposable wearable absorbent article in the form of a diaper shown with the body-facing surfaces facing the viewer, and shown with its chassis portion in a flattened condition, stretched out against tensile forces, process-induced contraction and material gathering caused by pre-strained elastic members in the chassis waist regions, barrier cuffs and leg bands;

FIG. 4 is a schematic cross section view of a disposable wearable absorbent article taken through the location indicated in FIG. 3;

FIG. 5 is a plan view of a disposable wearable absorbent article in the form of a diaper shown with the outer surfaces facing the viewer, and shown with its chassis portion in a flattened condition, stretched out against tensile forces, process-induced contraction and material gathering caused by pre-strained elastic members in the waist regions, barrier cuffs and leg bands;

FIG. 6 is a perspective view of a disposable wearable absorbent article in the form of a pant shown in a relaxed condition before application to a wearer;

FIG. 7 is a schematic plan view of a disposable wearable absorbent article in the form of a pant precursor structure shown with the chassis portion in a flattened condition, stretched out against tensile forces, process-induced contraction and material gathering caused by pre-strained elastic members that may be present in chassis waist regions, barrier cuffs and leg bands, prior to folding along a lateral axis and prior to joining of its respective side panel components to complete its final assembly into a pant;

FIG. 8 is a plan view of a disposable wearable absorbent article backsheet/outer cover shown with the outer surfaces facing the viewer, and shown in a flattened condition, stretched out against tensile forces, process-induced contraction and material gathering caused by pre-strained elastic members in the waist regions and leg bands;

FIG. 9 is a perspective schematic view of a die roller mechanism; and

FIG. 10 is a perspective schematic view of a die roller mechanism.

### DETAILED DESCRIPTION OF EMBODIMENTS

### Definitions and Conventions

For purposes of this description:

“Associating” and forms thereof, when used as a verb form in a phrase in which “package” is the subject and “articles” or “set” is the object, means, physically: containing, grouping, bundling, connecting, or otherwise holding a plurality of articles or plurality of sets of articles together in or with a single unit, for example, a package containing a plurality of disposable diapers as is often offered for retail sale as a unit.

“Chassis” means the laterally central structure of a wearable absorbent article that is adapted to be worn about the lower torso of a wearer, and is adapted to contain and support an absorbent body in proximity to a wearer's body. A chassis includes a topsheet and backsheet.

“Disposable”, when referring to an article, means that the article is not adapted to be effectively sanitarily cleaned or laundered in an ordinary household cleaning or laundering process and ordinary household equipment, and thereby is ordinarily unsuitable for sanitary and effective reuse so as to provide as-new intended functions and performance, following soiling by a wearer's exudates. By way of non-limiting examples, effective laundering may be frustrated or prevented, causing the article to be disposable, by inclusion of materials and/or construction: that do not retain their substantial as-new physical shape or structure through ordinary household laundering and drying so as to be effective as-new in reuse; that absorb aqueous liquids and cannot be sufficiently dried/dehydrated in ordinary household drying equipment and ordinary drying cycles so as to be effective as-new in reuse; that dissolve or substantially degrade in ordinary household laundering or drying, causing the article to be substantially damaged or rendered useless; and/or that cannot be effectively cleaned of exudate material through ordinary laundering, so as to be sanitary and otherwise acceptable for re-use.

“Film” means a skin-like or membrane-like layer of material formed of one or more polymers, which does not have a form consisting predominately of a web-like structure of consolidated polymer fibers and/or other fibers.

As used herein, the term “stretchable” refers to the property of a material that elongates, without substantial rupture or breakage, by at least 50% at a load of between 0.1 and 10 N/cm in the Hysteresis Test (as described herein). Rupture or breakage having a dimension less than 5 mm in any direction is not considered substantial rupture or breakage. However, ruptures through the structure having a dimension greater than 5 mm in any direction, breaks, ruptures or tears into two or more pieces, or breaks, ruptures or tears resulting in significant structural degradation which render the material unusable for its intended purpose, are considered substantial ruptures or breakage. A material that does not meet this definition for “stretchable” is considered “unstretchable.” A stretchable material may be elastic or extensible as defined herein.

As used herein, the term “elastic” refers to the property of a material that elongates, without substantial rupture or breakage, by at least 50% at a load of between 0.1 and 10 N/cm in the Hysteresis Test. Further, upon release of the load, the elastic material has a set less than or equal to 20% as measured according to the Hysteresis Test. For example, an elastic material that has an initial length of 25 millimeters can elongate to at least 37.5 millimeters (50% elongation) and, upon removal of the force, retract to a length of 27.5 millimeters, i.e., have a set of 2.5 millimeters (10% set), when subjected to the Hysteresis Test. It will be appreciated that this definition of elastic cannot be applied to materials such as individual elastic strands that do not have sufficient dimensions (e.g., not wide enough) to be properly subjected to the Hysteresis Test. As an alternative, such material is considered to be “elastic” if it can be elongated by at least 50% upon application of a biasing force, and return substantially to its original length exhibit less than 20% set) upon release of the biasing force.

As used herein, the term “extensible” refers to the property of a material that elongates, without substantial rupture or breakage, by at least 50% at a load of between 0.1 and 10 N/cm in the Hysteresis Test. Further, upon release of the load, the extensible material has a set greater than 20% as measured according to the Hysteresis Test. For example, an extensible material that has an initial length of 25 millimeters can elongate at least to 37.5 millimeters (50% elongation) and, upon removal of the applied force, retract to a length of 35 millimeters, i.e., have a set of 10 millimeters (40% set), when subjected to the Hysteresis Test.

“Lateral” (and forms thereof), with respect to a wearer, means along a direction generally transverse or across the direction extending from the front to the rear of the wearer, or vice versa, and horizontally with respect to a wearer's body when in a standing position. With respect to a component of a article, “lateral” (and forms thereof), means along a direction generally transverse or across the direction extending along the component as it would be properly situated on a wearer, from the front to the rear of the wearer, or vice versa.

“Longitudinal” (and forms thereof), with respect to a wearer, means along a direction generally extending from the front to the rear of the wearer, or vice versa, and vertically with respect to a wearer's body when in a standing position. With respect to a component of a article, “longitudinal” (and forms thereof), means along a direction generally extending along the component as it would be properly situated on a wearer, from the front to the rear of the wearer, or vice versa.

A “nonwoven”, or interchangeably, a “nonwoven web”, is a manufactured sheet or web of directionally or randomly oriented fibers which are first formed into a batt and then consolidated and bonded together by friction, cohesion, adhesion or one or more patterns of bonds created through localized compression and/or application of pressure, heat, ultrasonic or heating energy, or a combination thereof. The term does not include fabrics which are woven, knitted, or stitch-bonded with yarns or filaments. The fibers may be of natural or man-made origin and may be staple or continuous filaments or be formed in situ. Commercially available fibers have diameters ranging from less than about 0.001 mm to more than about 0.2 mm and they come in several different forms: short fibers (known as staple, or chopped), continuous single fibers (filaments or monofilaments), untwisted bundles of continuous filaments (tow), and twisted bundles of continuous filaments (yarn). Nonwoven fabrics can be formed by many processes including but not limited to meltblowing, spunbonding, spumelting, solvent spinning, electrospinning, carding, film fibrillation, melt-film fibrillation, airlaying, dry-laying, wetlaying with staple fibers, and combinations of these processes as known in the art. The basis weight of nonwoven fabrics is usually expressed in grams per square meter (gsm).

A “pre-strained” elastic member is an elastic member that has been placed into an article in a strained condition during manufacture of the article, such that relaxation and elastic contraction of the elastic member following manufacture causes web materials adjacent the elastic member to gather and form ruffles of material proximate the elastic member, aligned approximately transversely to the direction of the strain.

“User” means a parent, caregiver or other person who may apply an article to a wearer. Where the wearer is capable of donning the wearable absorbent article him/herself, the wearer is also a “user”.

“Wearer” means a person who wears a wearable absorbent article as described herein.

“Wearable absorbent article” or “article” means a diaper, absorbent training pant, absorbent incontinence pant or brief or similar article structured to be worn about the lower torso that has an absorbent structure comprising cellulose fiber, absorbent gelling material and/or superabsorbent polymer material, or other material, in addition to an outer cover, backsheet, outer pant or wrap, capable of absorbing and storing absorbed exudate fluid, including any article designed to be worn about the lower torso and to contain and/or absorb urine, feces, menses or any combination thereof, and includes but is not limited to baby or children's diapers or pants (of the “tape”-fastenable, otherwise fastenable, “pull-up” or any other variety), training pants (having permanently bonded side panels, detachable/reattachable refastenable side panels, belted or “balloon” waist configurations, or other configurations) and adult incontinence pants, incontinence briefs including absorbent structures, and the like.

For purposes herein, comparative measurements of a dimension of an article of a first set (or component of such article) and a comparable dimension of an article of a second set (or component of such article) are made under substantially similar test conditions, with each article or component to be measured extended, along the dimension to be measured, to the extent necessary to eliminate process-induced gathers of material that act to foreshorten or have the effect of foreshortening components or features of the article. For example, pre-strained elastic members disposed laterally in a waist region of an article, and pre-strained elastic members disposed substantially longitudinally in leg bands or barrier cuffs of the article, may foreshorten the article and components thereof laterally and longitudinally. Any such process-induced contraction is pulled out until gathers in materials are removed. The article may be secured to a flat, horizontal surface with clamps, weights, adhesive tape, or any other devices capable of holding the article in place with process-induced gathers pulled out. This approach of removing process-induced contraction on the article and/or components of interest prior to making measurements will be familiar to a person experienced in the analytical testing and measurement of absorbent article products. In some cases, an article may have component assemblies assembled with pre-strained elastic members, for example, a discrete waistband assembly, which may then be applied to a waist region of the article pre-strained by an amount less than the amount of pre-strain of the elastic members when the waistband assembly was made. The dimension of interest, however, may be that of the backsheet and not the waistband assembly. In such cases, it is not necessary to pull out all of the process-induced contraction from the waist band assembly, but rather, only from the backsheet. To enable measurement of dimensions of pant structures, side panels of pants that are permanently bonded together are forcibly separated or cut longitudinally, proximate their side seams or at laterally outermost hip areas; side panels of pants that are non-destructively detachable at seams are detached and separated at such seams.

It is necessary to differentiate between designed and/or specified differences in structures of articles of respective first and second sets associated by a package, and differences that may arise by happenstance as a result of normal process variations, which can result in articles manufactured to the same nominal dimensions and specifications nevertheless varying in structure to some extent. In some circumstances it may be difficult to determine which, and how many, of the articles associated by a package are designed to have a first structure and which, and how many, of the articles are designed to have a second structure, and it may be necessary to measure the dimension of interest of all of the articles associated by the package in order to determine whether the values cluster around two or more average values, with a statistically significant difference (95% confidence interval) between these clusters. For example, if the dimension of interest is the distance between the laterally outer edges of the outer leg band elastic members of articles associated by a package associating 20 articles in total, one can measure this dimension for all 20 of the articles, arrange the values in an ascending order with article number 1 having the lowest value and article number 20 having the highest value, and then plot these values on the y-axis and the article number on the x-axis. If the data cluster around two or more average values, with a statistically significant difference between these values, then it may be concluded that the variation in dimension from one set of articles to the other is by design, and within the scope of the claims herein which have no numerical limitations; if two of the average values exceed applicable numerical limitations in certain claims, and the package satisfies the other applicable elements as well, then the package falls within such claims. If, on the other hand, the data prove to be just randomly variable, such that no sets of respectively differing structures can be clearly identified, then one can conclude that the variation is due to normal process variations and is outside the scope of the claims. The foregoing approach also requires that each set of articles of the package with a designed-in structural difference from the other articles should comprise at least 3 and more preferably 5 articles. This would ensure that any outliers arising from random process variations are not treated as designed-in variations. A person of ordinary skill in the art may also use any other method to differentiate between a designed-in difference and a normal process variation.

In some cases, comparative measurements may be unduly difficult to obtain. For example, complexity of the construction of a feature being measured might make it unduly difficult to determine when all process-induced contraction has been removed by pulling, sufficient to enable comparative measurement. In such cases, comparison may be made between nominal and/or specified dimensions of the components of interest during manufacture, prior to assembly and/or prior to completed manufacture of the article and relaxation to process-induced contracted dimensions. The comparative claim limits herein are directed to intentional design, process and/or packaging choices to make articles having differing structures and associate the articles of the differing structures with the same package. Accordingly, if dimensions of finished articles associated by a package are unduly difficult to measure and compare, resort may be had to comparing nominal and/or specified dimensions of components upstream in the manufacturing process. For example, articles of a first set in a package may be manufactured from backsheet material intentionally cut to a first nominal and/or specified length, while articles of a second set in the package may be manufactured from backsheet material intentionally cut to a second nominal and/or specified length. Where numerical limitations are applicable as contained in some claims herein, if the differences in intended cut lengths fall within the numerical limitations, and the articles and packages otherwise have the recited elements, then they still fall within such claims, even if making comparative measurements of the finished articles proves unduly difficult. Conversely, where differences in dimensions between different articles associated with the same package is not the result of intentionally specified and differing designs, processes and/or packaging choices and selections, but rather, only the happenstance result of ordinary deviations that occur in manufacturing articles of the same intended design to the same nominal dimensions and specifications, such differences are not intended to be covered by the present claims.

For purposes herein, where a difference between a dimension of a feature of an article having a first structure and the comparable dimension of a similar feature of an article having a second structure is expressed as a percentage (e.g., at least about 10%, 15% or 20%), the percentage is calculated as the absolute value of the difference in the dimensions, divided by the smaller of the two dimensions, times 100%. For example, if an article having a first structure has a leg band width WLB₁ of 180 mm and an article having a second structure has a leg band width WLB₂ of 200 mm, the percent difference for purposes herein is:

[|180 mm-200 mm|/180 mm]×100%=11.1%

### Articles Generally

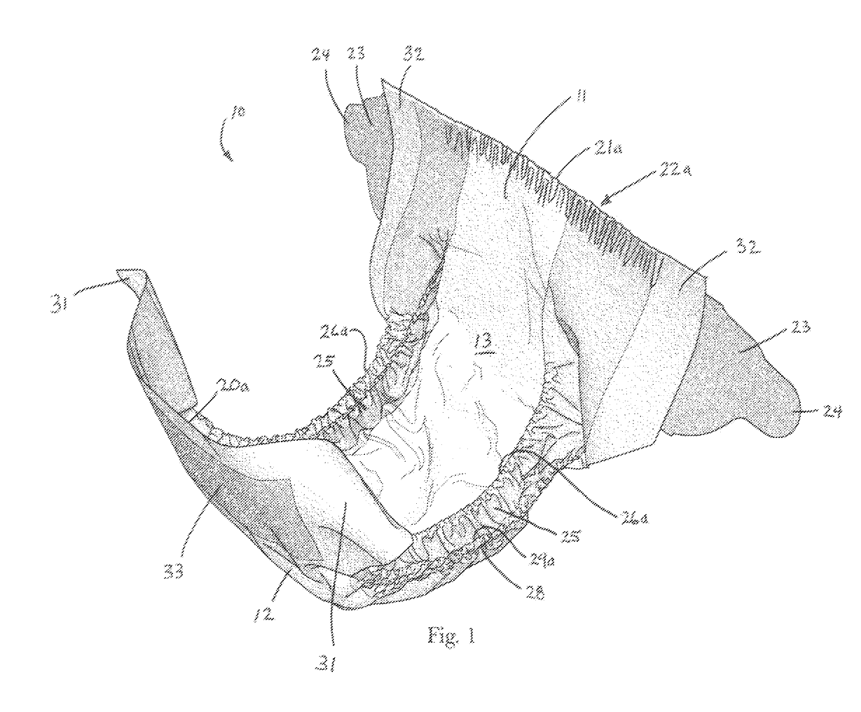

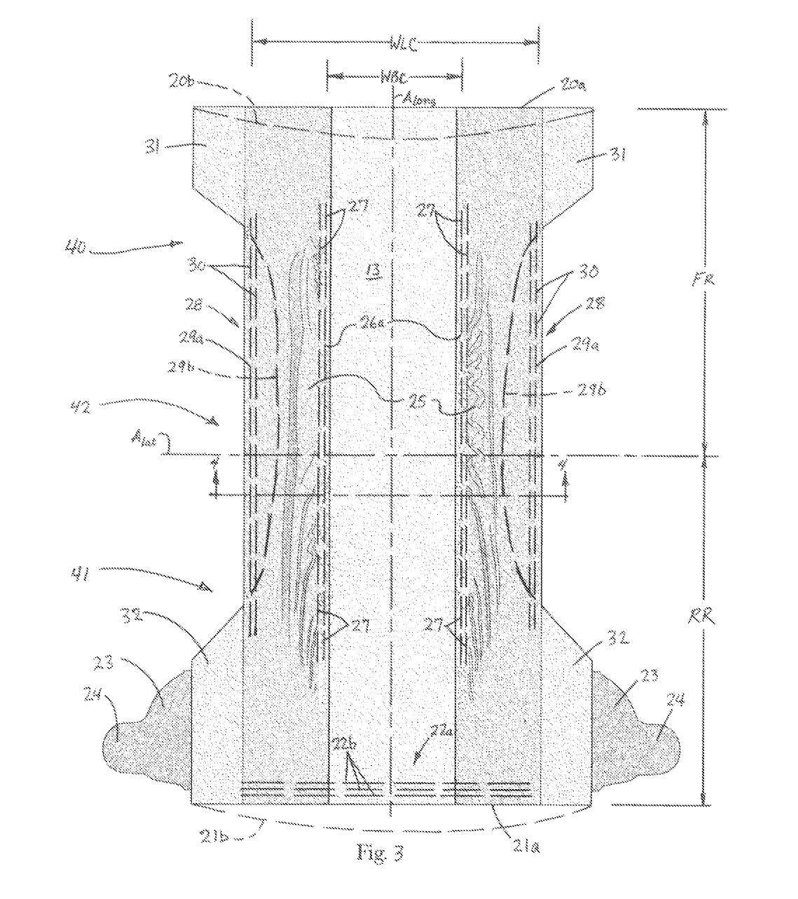

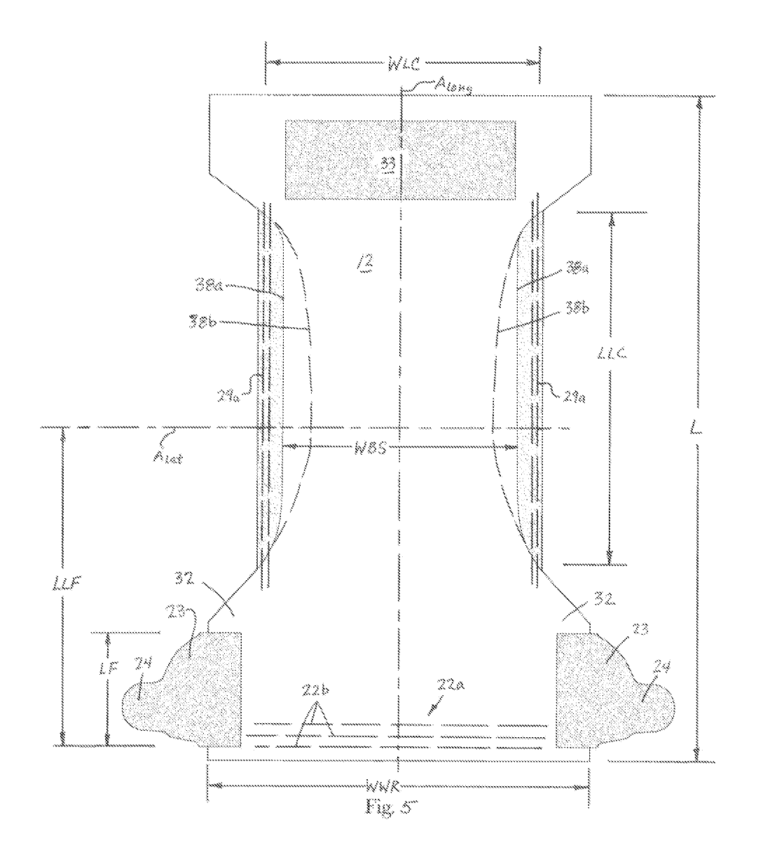

Referring to FIGS. 1-5, an article 10 generally may include a chassis 11 formed of topsheet 13, backsheet 12, and an absorbent core 14 disposed between the backsheet and the topsheet. The chassis 11 may comprise the main, longitudinally central body of the article 10. Chassis 11 has a front waist edge 20a and rear waist edge 21a. For reference herein, FIGS. 3 and 5 depict a longitudinal axis A_{long} equally dividing the width of the article, and a lateral axis Aₗₐₜ equally dividing the length of the article.

Topsheet 13 may be formed of a web material that is liquid pervious. Backsheet 12 may be formed of a web material or laminate of web materials that is liquid impervious, but may be air permeable or breathable. Absorbent core 14 may be enveloped or otherwise secured between the topsheet 18 and the backsheet 20. The chassis 11 may also include elasticized barrier cuffs 25 having barrier cuff edges 26a and elasticized leg bands 28 having leg band edges 29a, and a laterally elasticized waist region 22a. The article 10 may also comprise a fastening system, which may include at least one fastening member 23 having a fastening tab 24, and a landing zone 33. The fastening tab(s) 24 may have disposed thereon one component of a fastening system such as a hooks component of a hook-and-loop fastening system, and the landing zone 33 may include a second component of a fastening system, such as a loops component of a hook-and-loop fastening system.

One or more layers of the topsheet and/or backsheet may be formed of a nonwoven web as described below.

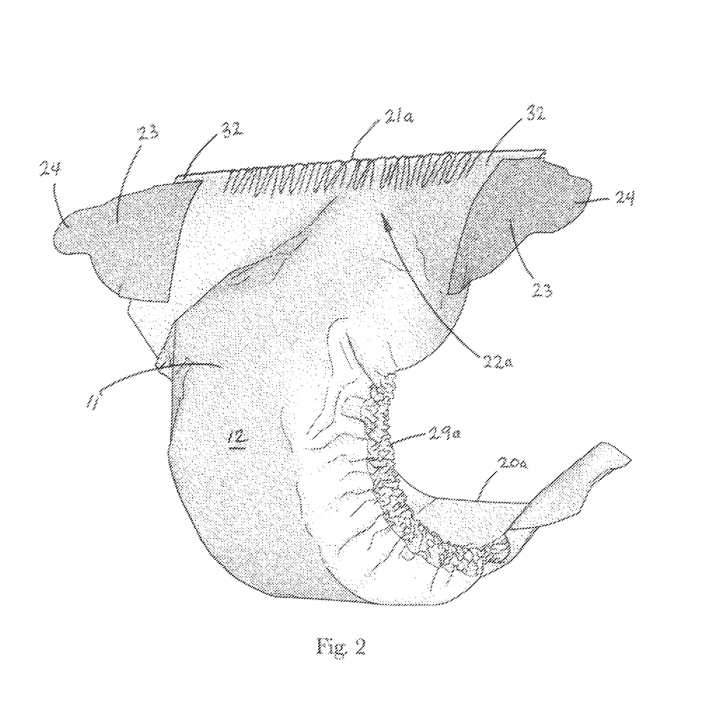

The barrier cuffs 25, leg bands 28 and elasticized waist region 22a may each include respective elastic members 27, 30 and 22b, which may be placed into the article during manufacture in a pre-strained condition, resulting in the gathers or ruffles of materials resulting from elastic contraction depicted in FIGS. 2 and 3. Elastic members 27 and 30 may be one or more strands or strips of elastomeric material, generally lying along a longitudinal direction within barrier cuffs 25 and leg bands 28. Elastic members 22b may also be one or more strands or strips of elastomeric material, generally lying along a lateral direction within elasticized waist region 22a.

One end portion of the article 10 may be configured as a front waist region 40 of the article 10. An opposite end portion of the article 10 may be configured as a rear waist region 41 of the article 10. An intermediate portion of the article 10 may be configured as a crotch region 42, which extends longitudinally between the first and second waist regions 40 and 41. The crotch region 42 may include from 33.3% to 50% of the overall length of the article 10, and each of waist regions 30, 32 may correspondingly include from 25% to 33.3% of the overall length of the article 10.

One or both of waist regions 40, 41 may include an elasticized waist region 22a as described above such that one or both tend to gather about the waist of the wearer to provide improved fit and containment. The crotch region 34 is that portion of the article 10 which, when the article 10 is worn, is generally positioned between the wearer's legs.

The article 10 may also include such other features including front and rear ear panels, waist cap features, elastics and the like to provide better fit, containment and aesthetic characteristics. Such additional features are described in, e.g., U.S. Pats. Nos. 3,860,003 and 5,151,092.

In order to apply and keep article 10 in place about a wearer's lower torso, the rear waist region 41 may be attached by the fastening members 23 to the first waist region 40 to form leg openings and a waist opening. Alternatively or in addition to inclusion of elasticized waist regions, fastening members 23 may be formed of material(s) that are elastic along in the lateral direction, providing additional stretch and contraction capability about the wearer's waist. When fastened properly on a wearer, the fastening system carries a lateral tensile load around the waist regions.

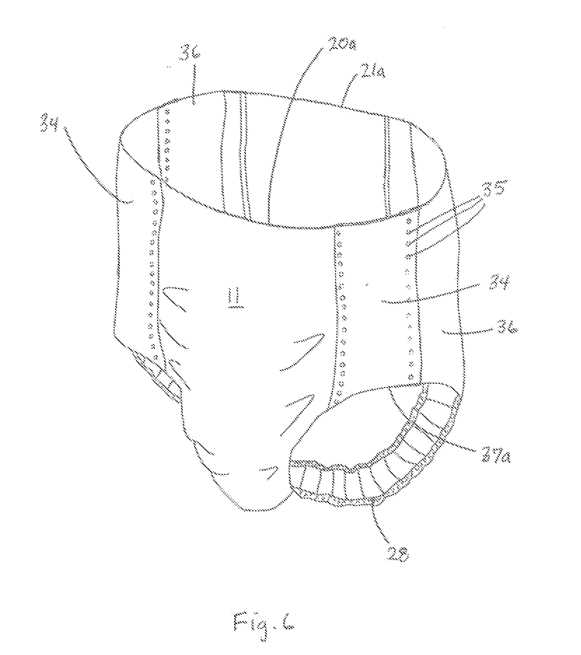

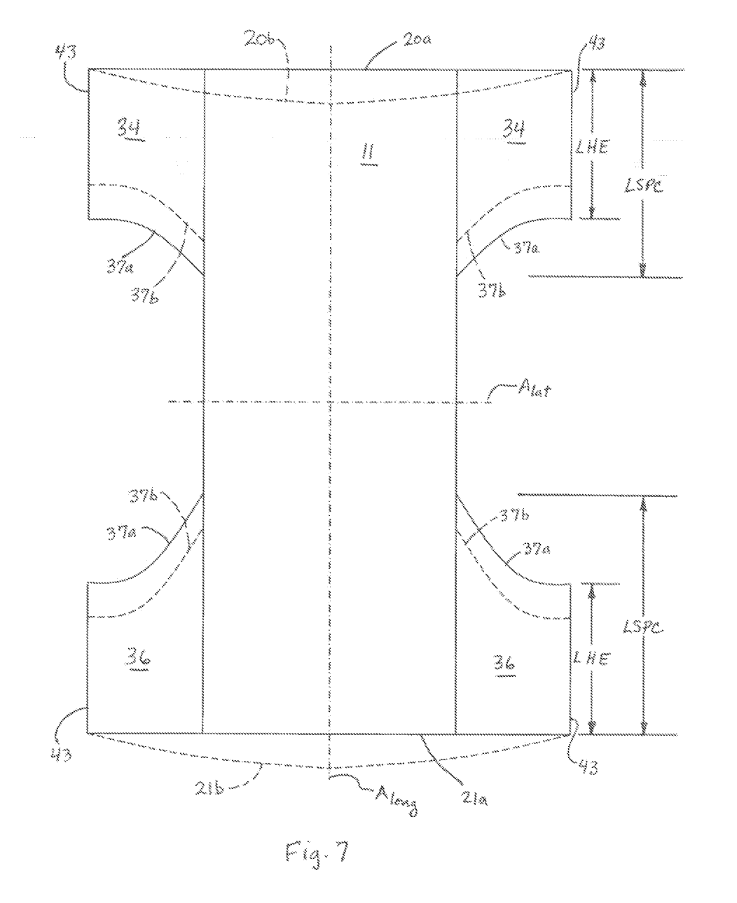

According to some examples, the article 10 may be provided with a re-closable fastening system (FIGS. 1-3, 5) in a “diaper” configuration, or may alternatively be provided in the form of a pant-type article (FIGS. 6, 7). When the absorbent article is diaper-type article, it may comprise a re-closable fastening system joined to the chassis for securing the article to a wearer. When the absorbent article is a pant-type article, the article may include one or more panels 34, 36 joined to the chassis and to each other to form a pant.

The fastening system and any component thereof may include any material suitable for such a use, including but not limited to plastics, films, foams, nonwoven, woven, paper, laminates, stretch laminates, activated stretch laminates, fiber reinforced plastics and the like, or combinations thereof. In some examples, the materials making up the fastening device may be flexible. In some examples, the fastening device may comprise cotton or cotton-like materials for additional softness or consumer perception of softness. The flexibility may allow the fastening system to conform to the shape of the body and thus, reduce the likelihood that the fastening system will irritate or injure the wearer's skin.

For unitary absorbent articles, the chassis 12 and absorbent core 14 may form the main structure of the article 10 with other features added to form the composite article structure. While the topsheet 13, the backsheet 12, and the absorbent core 14 may be assembled in a variety of well-known configurations, preferred article configurations are described generally in U.S. Pat. No. 5,554,145 entitled “Absorbent Article With Multiple Zone Structural Elastic-Like Film Web Extensible Waist Feature” issued to Roe et al. on Sep. 10, 1996; U.S. Pat. No. 5,569,234 entitled “Disposable Pull-On Pant” issued to Buell et al. on Oct. 29, 1996; and U.S. Pat. No. 6,004,306 entitled “Absorbent Article With Multi-Directional Extensible Side Panels” issued to Robles et al. on Dec. 21, 1999. In other examples, the absorbent core may be a discrete assembly which is attached within a backsheet structure at selected locations, exemplified in U.S. application Ser. No. 12/468,633, entitled “Attachment Areas for Wearable Absorbant Articles,” by Kline, the disclosure of which is incorporated by reference herein.

The topsheet 13 may be fully or partially elasticized and/or may be laterally and/or longitudinally foreshortened to create a void space between the topsheet 13 and the absorbent core 14. Exemplary structures including elasticized or foreshortened topsheets are described in more detail in U.S. Pat. No. 5,037,416 entitled “Disposable Absorbent Article Having Elastically Extensible Topsheet” issued to Allen et al. on Aug. 6, 1991; and U.S. Pat. No. 5,269,775 entitled “Trisection Topsheets for Disposable Absorbent Articles and Disposable Absorbent Articles Having Such Trisection Topsheets” issued to Freeland et al. on Dec. 14, 1993.

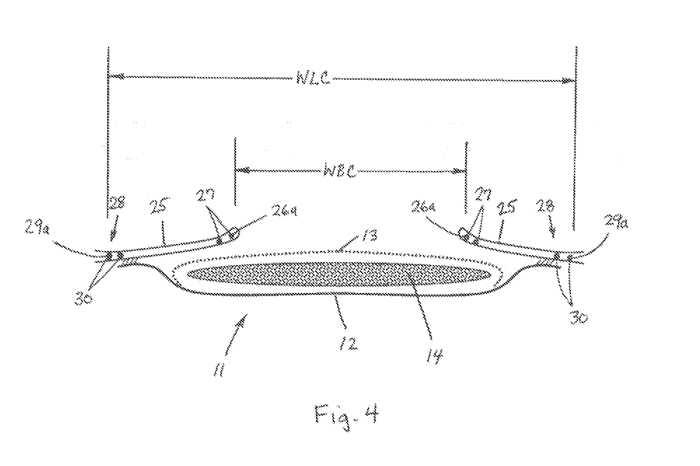

The backsheet 12 may be joined with the topsheet 13. The backsheet 12 may serve to prevent the exudates absorbed by the absorbent core 14 and contained within the article 10 from soiling other external articles that may come into contact the article 10, such as outer clothing, furniture, bedding, etc. Referring to FIG. 4, the backsheet 12 may be substantially impervious to liquids (e.g., urine) and may be formed of a laminate of a nonwoven and a thin polymeric film such as a thermoplastic film having a thickness of about 0.012 mm (0.5 mil) to about 0.051 mm (2.0 mils). The nonwoven component of the backsheet may be a nonwoven web as described herein. Suitable backsheet films include those manufactured by Tredegar Industries Inc. of Terre Haute, Ind. and sold under the trade names X15306, X10962, and X10964. Other suitable backsheet materials may include breathable materials that permit vapors to escape from the article 10 while still preventing liquid exudates from passing through the backsheet 12. Exemplary breathable materials may include materials such as woven webs, nonwoven webs, composite materials such as film-coated nonwoven webs, and microporous films such as manufactured by Mitsui Toatsu Co., of Japan under the designation ESPO and by EXXON Chemical Co., of Bay City, Tex., under the designation EXXAIRE. Suitable breathable composite materials comprising polymer blends are available from Clopay Corporation, Cincinnati, Ohio under the name HYTREL blend PI 8-3097. Other examples of such breathable composite materials are described in greater detail in PCT Application No. WO 95/16746, published on Jun. 22, 1995 in the name of E. I. DuPont. Other breathable backsheets including nonwoven webs and apertured formed films, and methods for making them, are described in U.S. Pat. No. 5,571,096 issued to Dobrin et al. on Nov. 5, 1996, and in U.S. application Ser. No. 12/534,353, entitled “Method for Making an Elastomeric Apertured Web,” by Qureshi et al.

In some examples, the backsheet of the present invention may have a water vapor transmission rate (WVTR) of greater than about 2,000 g/24 h/m2, greater than about 3,000 g/24 h/m2, greater than about 5,000 g/24 h/m2, greater than about 6,000 g/24 h/m2, greater than about 7,000 g/24 h/m2, greater than about 8,000 g/24 h/m2, greater than about 9,000 g/24 h/m2, greater than about 10,000 g/24 h/m2, greater than about 11,000 g/24 h/m2, greater than about 12,000 g/24 h/m2, greater than about 15,000 g/24 h/m2, measured according to WSP 70.5 (08) at 37.8° C. and 60% Relative Humidity.

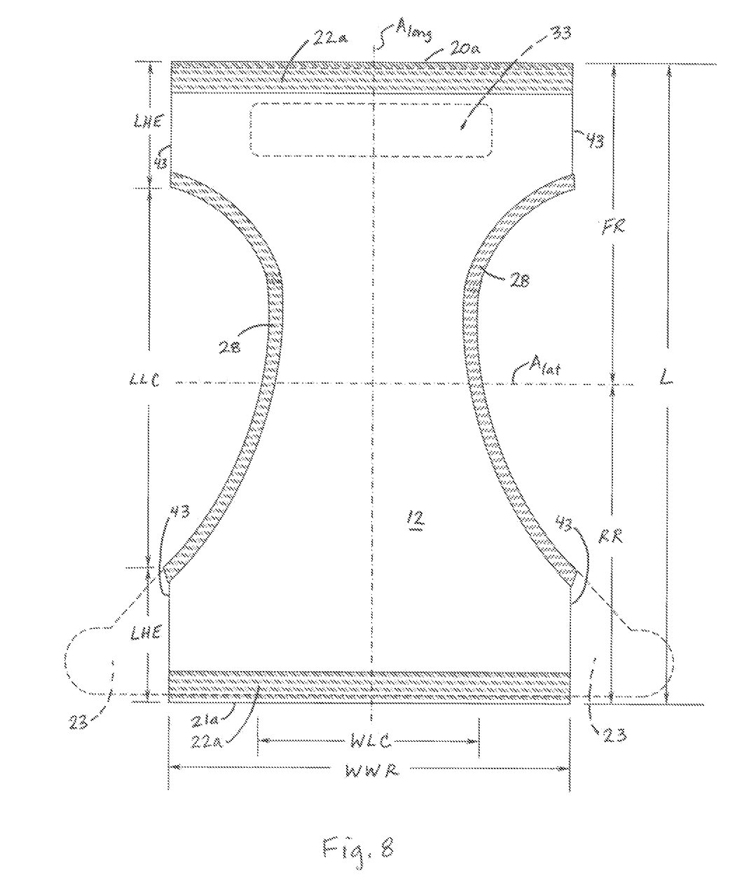

In some examples, a backsheet may be formed of a uni-directionally or bi-directionally, elastic web material comprising either a single layer of elastomeric film, or a laminate of an elastomeric film, scrim or network of elastomeric strands, and one or more layers of nonwoven web material which may be activated/incrementally stretched as known in the art, or gathered by means of formation of the laminate with the elastomeric material in a pre-strained condition. The elastomeric material may extend the entire length and width of the backsheet/outer cover, or it may be present only in portions of the backsheet, such as in the front and rear waist regions only and not in the crotch region, or in the four corners of the diaper only. Examples of a bi-directionally extensible material and backsheets made therefrom are described in U.S. application Ser. No. 11/599,829 entitled “Biaxially Stretchable. Outer Cover for an Absorbent Article,” by Autran et al., the disclosure of which is incorporated by reference herein. Referring to FIG. 8, in some examples, the entire backsheet 12 including side hip areas may be formed of such material, providing a stretchable backsheet that is elastically extensible in one or both of longitudinal and lateral directions. In such examples, a discrete absorbent core structure may be attached within the backsheet in any of the ways described in, for example, U.S. application Ser. No. 12/468,633, entitled “Attachment Areas for Wearable Absorbant Articles,” by Kline, the disclosure of which is incorporated by reference herein.

In some examples, discrete elastic leg bands and waist bands may be formed using, for example, elastomeric strands laminated between two layers of nonwovens or films, or combinations of the two as described in, for example, U.S. application Ser. No. 12/813,727, entitled “Prestrained Stretch Laminates,” by Langdon et al., the disclosure of which is incorporated by reference herein, and then attached to backsheet material along/about respective leg and waist openings by any appropriate bonding method, including adhesive, thermal, mechanical/compression, ultrasonic, etc. In a preferred embodiment, these waist and leg bands form the entire waist and leg hoops, that is they form waist and leg bands that entirely encircle the wearer's waist and legs. Still referring to FIG. 8, in a particular example, a backsheet/outer cover 12 may be cut from a web material, for example, a uni-directionally or bi-directionally, elastically extensible web material as described above, to form a pre-cursor to a pant structure having an hourglass-shape. Thereafter, discrete elastic and/or elasticized waist bands 22a and/or leg bands 28 may be attached along/about the leg and/or waist openings. Upon subsequent folding over approximately along a lateral line such as lateral axis Aₗₐₜ, and joining of the web material at hip edges 43 to form seams, a pant or brief structure may be formed. Alternatively, a backsheet/outer cover 12 may be formed as suggested in FIG. 8, and instead of being folded over and seamed at the hip edges to form a pant structure, backsheet/outer cover 12 may have fastening members 23 and landing zone 33 attached thereto, to form a diaper-type backsheet/outer cover structure. An absorbent core structure may be attached thereto/therewithin as described in, for example, U.S. application Ser. No. 12/468,633, entitled “Attachment Areas for Wearable Absorbant Articles,” by Kline, the disclosure of which is incorporated by reference herein.

Suitable nonwoven web materials useful for forming a backsheet nonwoven component, topsheet, or barrier cuff include, but are not limited to spunbond, meltblown, spunmelt, solvent-spun, electrospun, carded, film fibrillated, melt-film fibrillated, air-laid, dry-laid, wet-laid staple fibers, and other and other nonwoven web materials formed in part or in whole of polymer fibers, as known in the art. A suitable nonwoven web material may also be an SMS material, comprising a spunbonded, a melt-blown and a further spunbonded stratum or layer or any other combination of spunbonded and melt-blown layers, such as a SMS or SSMMS etc. Examples include one or more layers of fibers with diameters below 1 micron (nanofibers and nanofiber layers); examples of these rise in combinations of SMS, SMNS, SSMNS or SMNMS nonwoven webs (where “N” designates a nanofiber layer). Nonwovens including nanofibers or layers formed of nanofibers may be particularly preferred as components of barrier cuffs, since such nonwovens can be relatively breathable (air permeable) while being relatively liquid impermeable, as a result of hydrophobicity, and density and fineness of the fibers in the web. In some examples, permanently hydrophilic non-wovens, and in particular, nonwovens with durably hydrophilic coatings may be desirable for use as topsheet components, where liquid permeability is desired. Typically, the suitable non-woven is air permeable. Typically the suitable nonwoven is water or liquid permeable, but may also be water impermeable by reason of fiber size and density, and hydrophobicity of the fibers. Water or liquid permeability may be enhanced by treatments to render the fibers hydrophilic, as discussed below.

A nonwoven web may be formed predominately of polymeric fibers. In some examples, suitable non-woven fiber materials may include, but are not limited to polymeric materials such as polyolefins, polyesters, polyamide, or specifically polypropylene (PP), polyethylene (PE), poly-lactic acid (PLA), polyethylene terephthalate (PET) and/or blends thereof. Nonwoven fibers may be formed of, or may include as additives or modifiers, components such as aliphatic polyesters, thermoplastic polysaccharides, or other biopolymers (bio-based or renewable polymers). For purposes of providing a smooth, lubricious feel against the wearer's skin and reducing friction with the skin, nonwovens formed at least partly if not predominately of PE fiber components may be preferred for the topsheet, leg bands and/or barrier cuffs.

The individual fiber components of a nonwoven component may be monocomponent or multicomponent. Multicomponent fibers may be bicomponent, such as in a core-and-sheath or side-by-side arrangement. Often, the individual components comprise aliphatic polyolefins such as polypropylene or polyethylene, or their copolymers, aliphatic polyesters, thermoplastic polysaccharides or other biopolymers.

Further useful nonwovens, fiber compositions, formations of fibers and nonwovens and related methods are described in U.S. Pat. No. 6,645,569 to Cramer et al., U.S. Pat. No. 6,863,933 to Cramer et al., U.S. Pat. No. 7,112,621 to Rohrbaugh et al.; U.S. patent application Ser. Nos. 10/338,603 and 10/338,610 by Cramer et al., and 13/005,237 by Lu et al., the disclosures of which are incorporated by reference herein.

Some polymers used for nonwoven fiber production may be inherently hydrophobic, and they may be surface treated or coated with various agents to render them hydrophilic. A surface coating may include a surfactant coating. One such surfactant coating is available from Schill & Silacher GmbH, Böblingen, Germany, under the Tradename Silastol PHP 90. Generally it may be desired that nonwoven materials used as barrier cuff components be hydrophobic so as to be more resistant to passage of liquid therethrough (for purposes of containment), but that nonwoven materials used as topsheet components be hydrophilic so as to more readily admit passages of liquid (for purposes of drawing liquid from the wearer's skin and allowing it to pass through the material and into the absorbent core).

A nonwoven also may include other types of surface coating. In one example, the surface coating may include a fiber surface modifying agent that reduces surface friction and enhances tactile lubricity. Preferred fiber surface modifying agents are described in U.S. Pat. Nos. 6,632,385 and 6,803,103; and U.S. Pat. App. Pub. No. 2006/0057921. For purposes of providing a smooth, lubricious feel against the wearer's skin and reducing friction with the skin, nonwovens coated as described may be preferred for the topsheet, leg bands and/or barrier cuffs.

Any of the nonwoven types described herein may be used for the topsheet, barrier cuffs, backsheet outer layer, or loops component in a hook-and-loop fastening system of an absorbent article.

The absorbent core 14 generally may be disposed between the topsheet 13 and the backsheet 12. It may include one or more layers. A first layer may comprise a fibrous liquid acquisition/distribution layer (for receiving rapid liquid exudations and transporting and distributing the received liquid over the surface of the other core components), A second layer may be a liquid storage layer, and comprise particles of super absorbent polymer (SAP), also known as absorbent gelling material (AGM), which may contained within a liquid-permeable envelope structure. The storage layer (not specifically depicted) may include respective substrates, an absorbent particulate polymer material disposed on substrates, and a thermoplastic adhesive material disposed on and/or within the absorbent particulate polymer material and at least portions of the substrates as an adhesive for immobilizing the absorbent particulate polymer material on the substrates. Such systems are described in, for example, U.S. application Ser. Nos. 12/141,122; 12/141,124; 12/141,126; 12/141,128; 12/141,130; 12/141,132; 12/141,134; 12/141,141; 12/141,143; and 12/141,146. These applications generally describe absorbent core constructions that minimize or eliminate the need for and inclusion of airfelt or other forms of cellulose fiber in combination with particles of superabsorbent polymer (sometimes known as “substantially airfelt-free cores”). Preferred absorbent articles and cores are also described in U.S. application Ser. No. 12/141,122; U.S. Pat. Apps. Pub. Nos. 2004/0167486A1 and 2004/0162536; and PCT Pub. No. WO 2009/060384.

Package

Undesirable marking, irritation and/or chafing of a wearer's skin may be caused or exacerbated by repeated wearing of successive disposable wearable absorbent articles having elasticized leg openings, leg bands, waist openings and/or waist bands of substantially unchanging dimensions. This is because such wear may result in repeated, continuous, encircling, concentrated, localized contact, and/or elevated contact pressure, at areas of the wearer's skin about the legs and/or waist, resulting from the presence of elastic members in the article. The risk of skin irritation may be increased with extended wear of an article loaded with urine, which makes the article heavier and bulkier, and increases stresses within the article and pressure at locations where the article contacts the wearer's skin.

To reduce the likelihood of skin marking, irritation and/or chafing, a plurality of respective sets of articles having at least two differing structures may be used when it is desired to substantially continuously keep a wearer wearing such articles over extended periods of time. Varying the structures of articles successively applied to a wearer such that features such as cuffs, leg bands, leg edges, waist bands and/or waist edges contact the wearer at locations that vary in one or more locations with a change of an article, may reduce the likelihood of marking, irritation and/or chafing. This is because the length of time over which the same areas of the wearer's skin are continuously contacted by such features is reduced, when successive articles cause areas of skin contact to change with a change of articles (e.g., diaper change).

Conceivably, a user might discover the problem of potential irritation and/or chafing associated with successive application of substantially identical articles to a wearer. The user might, further, discover that he or she can purchase supplies of differing types, designs, sizes or brands of articles, and then intentionally mix them in sequence when changing the articles on the wearer. However, this would impose a requirement for additional levels of attention and effort upon the user, which may be unwelcome in many situations, e.g., to busy parents or other caregivers. It may also require the user to purchase supplies of differing brands of articles, which the user may find inconvenient or undesirable.

It may be desirable, therefore, to include, with a package associating a total quantity of articles, a plurality of sets of articles together constituting the total quantity. The articles in each set may have a structure common to the set, while the structures may differ from set to set. All articles in all sets included with the package, however, may have a common wearer size design and designation, thus, size design and designation may be common for the total quantity.

The number, i.e., total quantity, of articles associated by the package may be selected to constitute at least several days' supply of articles. For example, a package may associate a total quantity of 18 or more articles, more preferably 24 or more articles, and even more preferably 30 or more articles.

The numbers of articles within the differing sets constituting the total quantity may be selected to be practicable and effective for the purpose described herein, i.e., varying the placement and fit of articles about the wearer's waist and legs. A first set of articles constituting a first portion of the total quantity may have a first structure, and a second set constituting a second portion of the total quantity may have a second structure. A package may associate at least two sets of articles, the articles within each set having a common structure, the structure of the first set differing from that of the second set. However, it may be desirable, for greater effect at varying article placement and fit about waist and leg openings, to include more than two sets, i.e., more than two different structures, for example, three different structures, four different structures, five different structures, or even six different structures.

For greatest convenience and effect of varying the placement and fit of articles about the wearer's waist and legs, the respective numbers of the articles in the sets may have a ratio that promotes relatively consistent variation in application of differing article structures to the wearer. Thus, the ratio of the number of articles in a first set to the number of articles in a second set, the sets each constituting portions of the total quantity associated by the package, may be from 1:2 to 1:1. For example, in a package associating a total quantity of 18 articles, a first set may include 9 articles and a second set may include 9 articles, where only two sets are present, the ratio being 1:1. In another example where three sets are present and constitute the total quantity, there may be six in each set (whereby the ratio between numbers in each group of two sets is 1:1), or in another alternative example within the total quantity of 18 and the ratios set forth above, three in a first set, three in a second set, and six in a third set (whereby the ratio between numbers of articles in the first and second sets is 1:1, and the ratio between numbers of articles in the first and third sets, and in the second and third sets, is 1:2).

The articles within the respective sets constituting the total quantity associated by the package need not be homogeneously grouped together. For user convenience in effectuating the method embodied in the invention, it may be desirable for the articles in the differing sets to be intermingled, so that the user encounters articles having the differing structures sequentially as he or she accesses the package for each change of articles. For example, in a package associating a total quantity of 18 articles, with a first set including 9 articles and a second set including 9 articles, the articles may be stacked such that the stack alternates from an article of the first set to an article of the second set, such that the user encounters the alternating structures as the user changes articles on the wearer. This intermingled arrangement may further ease the burden on the user, by making it unnecessary for the user to have to remember to vary article structures when changing articles.

Another way to aid the user in effecting the method embodied by the invention described herein is to differentiate differing structures by differing user-perceivable indicia. For example, articles of a first set and first structure may bear a first visible indicium common to all articles of the first set. Articles of a second set and second structure may bear a second visible indicium common to all articles of the second set, and conspicuously and noticeably different, to a user, from the first indicium. In a particular illustrative example, all articles in a first set may have a numeral “1” conspicuously imprinted on the backsheets thereof, while all articles in a second set may have a numeral “2” conspicuously imprinted on the backsheets thereof. In another example, articles of the first set may bear a first print color scheme, or design, and articles of the second set may bear a second print color scheme, or design. It will be appreciated that any number of effectively differentiating indicia may be used. Such differentiating indicia may serve to distinguish articles of differing sets to the user, so that the user, for example, may readily know when changing articles on a wearer, to replace an article bearing a first indicium with an article bearing a second indicium.

In order to effectuate variance of article placement and fit about leg openings, the structure of an article in a first set may differ from a structure of an article in a second set in a number of ways.

Referring to FIGS. 3-5, an article may have leg bands 28 having leg hand edges 29a, and barrier cuffs 25 having barrier cuff edges 26a. As used herein, a “leg band edge” is the laterally outer edge, at the lateral axis Aₗₐₜ, of any leg band elastic member 30 forming a leg band. Similarly, a “barrier cuff edge” is the laterally innermost, or uppermost (relative FIG. 4), edge, at the lateral axis Aₗₐₜ, of any barrier cuff elastic member 27 included to elasticize the body-hugging edge of the barrier cuff. Leg bands 28 may have a length LLC.

Referring to FIGS. 3 and 4, the it and placement of leg band edges 29a about a wearer's logs can be varied by varying the leg band width WLB between the leg band edges 29a. Width WLB may be varied from an article structure of a first set to an article structure in a second set by at least about 10%, 1.5%, 20% or more. For purposes herein leg band width WLB is measured at the lateral axis Aₗₐₜ.

The fit and placement of leg band edges 29a about a wearer's legs, and fit and placement of barrier cuff edges 26a in the wearer's crotch region, can be varied by varying the barrier cuff width WBC between the barrier cuff edges 26a. In order to most likely be effective for the purposes described herein, width WBC may be varied from an article structure of a first set to an article structure in a second set by at least about 10%, 15%, 20% or more. For purposes herein barrier cuff width WBC is measured at the lateral axis Aₗₐₜ.

Still referring to FIGS. 3 and 4, the fit and placement of leg band edges 29a about a wearer's legs can also be varied by varying the leg band length LLC. In order to most likely be effective for the purposes described herein, leg band length LLC may be varied from an article structure of a first set to an article structure in a second set by at least about 10%, 20%, 30%, 40%, or more. For purposes herein, the “length” of a leg band may be determined by placing marks at the respective longitudinal extents of gathered material having process-induced contraction and gathers about laterally outermost leg band elastic members 30 (while the article is in a relaxed state), pulling out the process-induced contraction as described hereinabove, and then measuring the longitudinal distance between the marks.

In another alternative, the fit and contact pressure of leg bands 28 may be varied from a structure of a first set to a structure of a second set by varying the amount of tension in the leg bands imparted by the leg band elastic members 30 when the article is worn. This may be effected by, for example, varying the number of elastic members included in the leg band structure, varying the size of the elastic members included in the leg band structure, or varying the material(s) of which the elastic members included in the leg band structure are formed. In one particular alternative, a first structure may have one or more leg band elastic members, and a second structure may omit leg band elastic members, the second structure being designed to rely primarily on barrier cuffs with one or more barrier cuff elastic members 27, for sufficient exudate containment capability.

Still referring to FIG. 3, another way in which the fit of leg bands 28 and leg band edges 29a may be varied is by varying the profile of the leg band edges. As depicted FIG. 3, leg band edges 29a are relatively longitudinally straight. Leg bands 28 may be manufactured differently, however, to have a differing profile. For example, leg bands 28 may be manufactured so as to have a curved profile such as a concave or other curved profile, e.g., leg band edges 29b having a concave profile.

Another way of varying fit and contact points of the article about the legs may be in varying the dimensions and/or profile of the backsheet film in the crotch region, proximate the leg openings. In many articles of the type described herein the backsheet is formed at least in part of a polymeric film layer component. This film layer component may or may not be designed with elastic stretch capability. In either case, backsheet film layer component imparts a substantial portion of the mechanical properties to the chassis, and the locations of its edges about the leg openings affect the way in which the materials about the leg openings fit about and contact the wearer. Referring to FIG. 5, a backsheet film may have leg opening edges 38a, and the backsheet film may have a backsheet film crotch width WBS measured at the lateral axis Aₗₐₜ. This width may be varied from a first structure to a second structure. In order to most likely be effective for the purposes described herein, backsheet film crotch width WBS may be varied from an article structure of a first set to an article structure in a second set by at least about 1.0%, 15%, 20% or more. In another alternative, the profile of the backsheet film leg opening edges may be varied from a first structure to a second structure. For example, a first structure may have backsheet film leg opening edges 38a having a first profile, and a second structure may have backsheet film leg opening edges 38b having a second profile.

Still another way of varying the fit and skin contact points of the article may be used to affect the locations of contact pressure in the wearer's hip areas. Referring again to FIG. 5, in order to be made most likely effective for the purposes described herein, the fastening member length LF may be varied from a first structure to a second structure by at least about 10%, 15%, 20% or more. (For purposes herein, the “fastening member length” is measured as the longitudinal length of the material forming the fastening member, along the location where the material forming the fastening member meets or passes over or under the polymer film forming the backsheet. In another alternative, in order to most likely be effective for the purposes described herein, the longitudinal location LLF of the top edge of the fastening member relative the chassis may be varied from a first structure to a second structure by at least about 10%, 15%, 20% or more, where, for purposes herein, LLF is measured from the lateral axis Aₗₐₜ.

Yet another way of varying the fit and skin contact points at the locations of contact pressure in the wearer's hip areas is to vary the profile or shape of the fastening members, from a first structure to a second structure.

Referring to FIGS. 6, 7 and 8, in articles configured as pants, the fit about leg openings may be varied by varying the size(s) and/or shape(s) of the rear and front side panels 34, 36, or the leg band 28 profiles. In pants, the leg openings are defined by the lower edges of panels 34, 36, or otherwise by leg bands 28. Thus, for example, side panels of articles of a first structure may have lower edges 37a, while side panels of articles of a second structure may have lower edges 37b. Correspondingly, in order to most likely be effective for the purposes described herein, the hip edge length LHE, of a panel 34 and/or 36 (e.g., as shown in FIG. 7), or otherwise of hip edges of a unitized backsheet/outer cover (e.g., as shown in FIG. 8), of a first structure may differ from that of a second structure by at least about 10%, 15%, 20% or more. Similarly, in order to most likely be effective for the purposes described herein, the chassis edge length LSPC of a panel 34 and/or 36 (e.g., as shown in FIG. 7) of a first structure may differ from that of a second structure by at least about 10%, 15%, 20% or more. (As used herein, the “chassis edge length” LSPC of a side panel is measured along a longitudinal line beginning at the point at which material forming the side panel but not the chassis (which may be, e.g., elastic material) extends laterally outwardly and away from the longitudinal edge of the polymer film component of the backsheet.)

Referring to FIG. 8, an article of a first structure may differ from an article of a second structure by having a differing leg band 28 profile. For example, articles of a first structure may have a brief-style leg band profile, while articles of a second structure may have a bikini-style leg band profile, or alternatively, a short-style leg band profile. It will be appreciated that any number of combinations of leg band profiles are possible to effect differences in the ways articles of a first structure and articles of a second structure fit about a wearer's legs.

Referring to FIGS. 6, 7 and 8, another manner in which a pant-type article of a first structure may differ from an article of a second structure may be the manner and extent to which material(s) along hip edges 43 are bonded to each other, and/or to chassis 11. Side panels 34, 36 may be bonded to each other by one or more bonds 35. Bonds 35 may be weld-like bonds formed by one or more of concentrated pressure, thermal energy and mechanical intermeshing and/or fusing of the materials of the respective side panels 34, 36, at bonding sites or zones. Alternatively, bonds 35 may be formed by one or more deposits of adhesive adhering materials of the side panels 34, 36 together. The one or more bonds 35 occupy a longitudinal length (not specifically shown) along a side seam joining panels 34, 36. Similarly, such bonds may join either or both of side panels 34, 36 to chassis 11 along a chassis seam, and occupy a longitudinal length along the chassis. In order to most likely be effective for the purposes described herein, these longitudinal bonding lengths along the chassis seam and along the side seam may be varied from a first structure to a second structure by, for example, at least about 10%, 15%, 20% or more. Even if the dimensions of the side panels and chassis are otherwise the same from structure to structure, varying the bonding length at the side and/or chassis seam(s) from a first structure to a second structure causes the location of contact pressure between the side panels and the wearer's skin to change, effected by a change from an article of the first structure to an article of the second structure.

As with leg openings, in order to effectuate variance of article placement and fit about the waist opening, the structure of an article in a first set may differ from a structure of an article in a second set in a number of ways.

Referring again to FIGS. 3, 5 and 8, an article may have a rear waist region width WWR, an overall length L, a front rise FR, and a rear rise RR. As used herein, the “rear waist line width” is the widest width of the polymer film component of the backsheet in the rear region, not including fastening members 23. The “front rise” and “rear rise” are measured from the lateral axis Aₗₐₜ to the greatest longitudinal extent of a lateral waist edge (e.g., 20a, 21a) of any component in the front region 40, and rear region 41, respectively.

One way of varying the longitudinal location of materials about the waist opening with respect to a wearer is by varying the overall length L of the article. In order to most likely be effective for the purposes described herein, article length L may be varied from an article structure of a first set to an article structure in a second set by at least about 5%, 7.5%, 10% or more. For purposes herein, article length L is the greatest longitudinal length measurable between the lateral edges of any components of the article.

Another way of varying the longitudinal location of materials about the waist opening with respect to a wearer is by varying either or both of the front rise FR and the rear rise RR. In order to most likely be effective for the purposes described herein, front rise FR and/or rear rise RR may be varied from an article structure of a first set to an article structure in a second set by at least about 5%, 7.5%, 10% or more.

Still another way of varying the location of materials about the waist opening with respect to the wearer is by varying the profile(s) of one or both of the waist edges. For example, as suggested in FIG. 3, an article of a first structure may have a front waist edge 20a that has a substantially straight lateral profile. An article of a second structure may have a front waist edge of a different profile such as a concave profile shown at 20b. Similarly, an article of a first structure may have a rear waist edge 21a that has a substantially straight lateral profile, while an article of a second structure may have a rear waist edge of a different profile such as a convex profile shown at 21b. It will be appreciated that the waist edge profiles can be varied in a number of ways. However, it may be preferred that a front waist edge profile if other than a straight profile, has a concave or otherwise longitudinally cut-in or scooped profile, while a back waist edge profile has a convex or otherwise longitudinally outward-projecting profile. For identical structures of the same set, the respective front and rear waist edge profiles may be complementary, in that they follow the same cut profile; this provides for efficiency in usage of materials and elimination of waste in manufacturing articles of the same structure. Additionally, it may be preferred that a front waist edge have a concave or otherwise longitudinally cut-in or scooped profile, for purposes of improved comfort to the wearer through better conformity to the contours of the wearer's abdomen (the front waist edge profile sometimes known as a “belly cut”).

Still another way of varying the fit and location of skin contact pressure about the waist opening is by varying the longitudinal location of one or more of waist elastic members 22b. In order to be made most likely effective for the purposes described herein, relative lateral axis Aₗₐₜ, the longitudinal location of one or more of waist elastic members 22b may vary from a first structure to a second structure by at least about 5%, 7.5%, 10% or more.

In another alternative, the fit and skin contact pressure of elasticized waist region 22a may be varied from a first structure to a second structure varying the amount of tension imparted by the waist elastic members 22b when the article is worn. This may be effected by, for example, varying the number of waist elastic members 22b included in the elasticized waist structure, varying the size(s) of the waist elastic members 22b included in the elasticized waist structure, or varying the material(s) of which the waist elastic members 22b included in the elasticized waist structure are formed. In one particular alternative, a first structure may have one or more waist elastic members, and a second structure may omit waist elastic members, the second structure being designed to rely primarily on lateral elastic stretch and contraction capability of the fastening members 23 (FIGS. 3, 5) and/or side panels 34, 36 (FIG. 7), or even an elastically stretchable backsheet/outer cover alone, for lateral elasticized fit about the waist opening.

The overall waist opening skin contact pressure can also be varied from a first structure to a second structure by varying the rear waist region width WWR

From the foregoing it will be appreciated that many permutations of combinations of the variances from a first structure to a second structure as described above may be used to effect variance in the placement and fit of articles about the wearer's waist and legs. To illustrate, any combination of the variances between a first structure and a second structure summarized below may be manifest in differing first and second sets of articles associated by a package:

| Leg Opening/Crotch Fit Variance | Waist Opening Fit Variance |
|---|---|
| Leg band width WLB | Overall length L |
| Barrier cuff width WBC | Front rise FR |
| Leg band length LLC | Rear rise RR |
| Presence and number of leg band elastic | Front and/or rear waist edge |
| members | profiles |
| Size of leg band elastic members | Longitudinal location of one or |
| Material(s) of which leg band elastic | more of waist elastic members |
| members are formed | Presence and number of waist |
| Leg band edge profile | elastic members |
| Backsheet film crotch width WBS | Size of waist elastic members |
| Backsheet film leg opening edge profile | Material(s) of which waist |
| Fastening member length LF | elastic members are formed |
| Fastening member top edge | Rear waist region width WWR |
| location LLF | |
| Fastening member profile/shape | |
| Hip edge length LHE (pant) | |
| Side panel chassis edge length | |
| LSPC (pant) | |
| Side seam longitudinal bonding length | |
| Side panel longitudinal bonding length | |
| (chassis seam) | |

One way in which articles of differing structures may be manufactured by one manufacturing line for on-line packaging, is to effect differing cutting profiles of component materials in sequential way, within the same line. For example, web materials are typically introduced to an article manufacturing line in the form of a supply roll of the material having a roll width, which is then drawn into the line off the roll for downstream processing into one or more components of an article. The web is conveyed through the line in a machine direction and undergoes various processes that may include cutting, slitting, bonding, gluing, laminating, folding, imprinting, etc., during manufacture of finished articles from incoming materials such as a web material. It is typically necessary to cut or slit such materials in the cross direction to sequentially form components of discrete articles. In some lines such materials also may be cut or slit generally along the machine direction to remove rough machine-direction edges, and/or to effect desired cutout profiles in components. Rolls of web materials may include backsheet components such as nonwovens, films and/or laminates thereof, internal components such as nonwovens for cuffs, dusting layers, absorbent core envelopes, topsheet components such as nonwovens and films, etc.

In one example of a typical article manufacturing line, backsheet web material is drawn into the line from a supply roll. At one station in the process the backsheet web material may be conveyed and/or drawn into the nip of a die cutting mechanism. A die cutting mechanism may include a die roller having one or more cutting blades mounted onto or into the cylindrical surface thereof, with cutting edges directed radially outwardly of the axis of the die roller. The die roller may be situated in contact with an opposing anvil roller, such that the cutting edges, situated to effect desired cuts of the web material, contact and roll along the surface of the anvil roller.

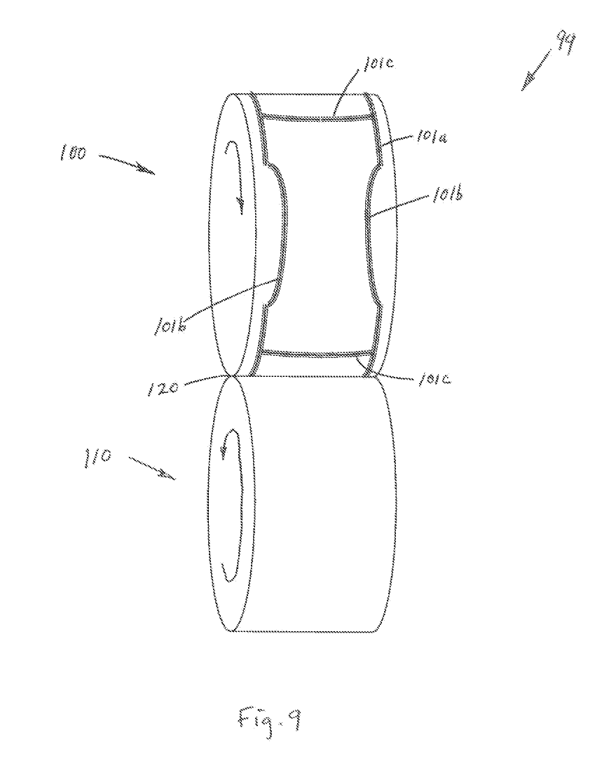

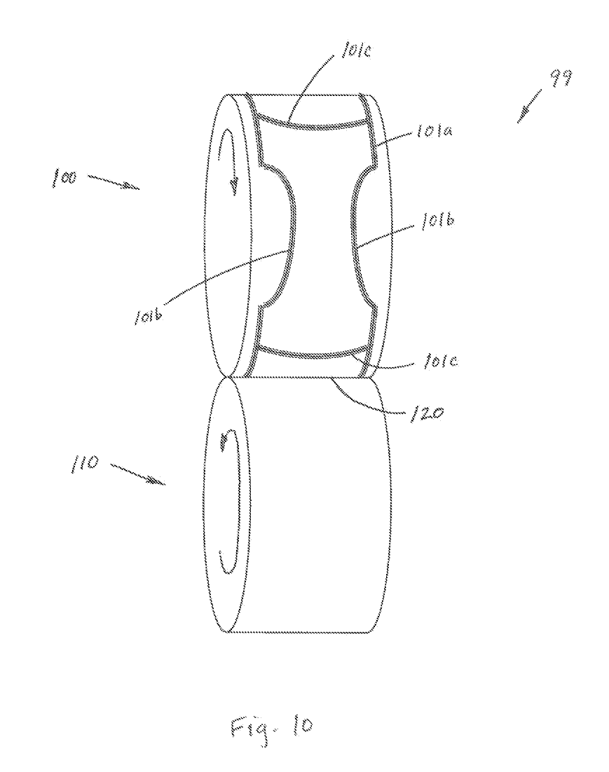

FIGS. 9 and 10 schematically depict one or two die roller mechanisms 99 each having a die roller 100 and an anvil roller 110 disposed in operable contact at a nip 120. Die roller 100 may have one or more cutting blades 101 mounted at or about the cylindrical surface thereof. Cutting blade portions 101a may effect longitudinal edge cuts of a backsheet profile which include leg cutout portions 101b, proximate the leg opening and hip areas of the finished article. Cutting blade portions 101c may effect lateral edge cuts of a backsheet profile which are proximate the waist edges of the finished article.

FIGS. 9 and 10 reflect manufacturing of article backsheets in a longitudinal direction, i.e., the machine direction is generally parallel with the longitudinal direction of the finished article. It will be understood, however, that some article manufacturing lines are arranged such that the machine direction is generally perpendicular to the longitudinal direction of the finished articles being manufactured, such that backsheets are sequentially cut side-from-side, rather than cut waist edge from waist edge as suggested in FIGS. 9 and 10. If backsheets or other components are manufactured such that the machine direction is perpendicular to the longitudinal direction of the finished articles, it will be appreciated that the cutting blades 101 of the associated die roller mechanism(s) need merely be arranged accordingly.

In order to manufacture articles of two or more differing structures on a single line, wherein the difference in structure is reflected in differing dimensions and/or cutout profiles of a web material component such as a backsheet, one or more die roller mechanisms such as depicted in FIGS. 9 and 10 may be used. In one example, two die roller mechanisms may be arranged in series wherein in a first die roller mechanism effects a first cutout profile and leaves an uncut space upstream of the first cutout profile, and then a second die roller mechanism downstream of the first die roller mechanism effects a second cutout profile in the space left uncut by the first die roller mechanism.

In another alternative, cutting blades on a single die roller mechanism may be arranged to singly and sequentially effect two or more cutout profiles sequentially. Referring to FIGS. 9 and 10, for example, FIG. 9 may depict cutting blade(s) 101 arranged on one portion of the die roller to effect a first cutout profile, while FIG. 10 may depict cutting blades(s) 101 arranged on another portion of the same die roller to sequentially effect a second cutout profile. The example in FIG. 9 depicts a more brief-like cutout profile, while the example in FIG. 10 depicts a more bikini-like cutout profile, with deeper leg cutouts and waist line contours. It will be appreciated that more than two blade arrangements to effect more than two cutout profiles may be arranged on a single die roller, as permitted by the size of the die roller relative the size of the cutout profiles needed.

In differing article component cutouts produced sequentially on a single manufacturing line, it is also possible to apply elastic members and/or discrete component assemblies including elastic members, as needed along the differing profiles. For example, the methods and equipment for applying elasticized strip materials described in co-pending U.S. application Ser. No. 12/363,002 by Eckstein et al., which is incorporated fully herein by reference, may be employed to apply elasticized strip leg band materials along leg cutout edges of varying profiles. In another example, the methods and equipment for applying elastic members along varying profiles described in U.S. Pat. No. 5,525,175 to Blenke et al., which is incorporated fully herein by reference, may be employed. It will be appreciated that one or both of these references describe equipment that can effect movement of strand or strip members in a cross direction as they are applied to a web moving in a machine direction, and this equipment may be driven by servo motors to effect varying ranges and speeds of cross-direction motion. Since servo motors may be controlled via programming, it will be appreciated that such equipment may be arranged and controlled to apply elastic and/or elasticized members along sequentially varying cutout profiles.

In a preferred embodiment, an elastic strip of material, for example a leg band, is applied to the outercover/backsheet in two different profiles, corresponding to the leg hoop shapes desired in two adjacent diapers in the package. These leg hoop shapes are then registered with corresponding die cut shapes for the leg opening, in order to deliver adjacent products in the bag with different leg band shapes. If desired, this approach can be extended to three or more different leg hoop shapes in the bag.

### Hysteresis Test

The following test methods utilize a commercial tensile tester (e.g., from Instron Engineering Corp. (Canton, Mass.), SINTECH-MTS Systems Corporation (Eden Prairie, Minn.) or equivalent) interfaced with a computer. The computer is used to control the test speed and other test parameters and for collecting, calculating, and reporting the data. The tests are performed under laboratory conditions of 23° C.±2° C. and relative humidity of 50%±2%. The samples are conditioned for 24 hours prior to testing.

1. Select a 2.54 cm (width), 7.62 cm (length) sample of the material for testing. In some cases, if it is not be possible to get a 2.54 cm×7.62 cm sample, a smaller sample may be used, but a gage length of 25 mm must still be used. If the sample is activated or includes an activation portion, the length of the sample is taken in the direction of activation.

2. Select the appropriate jaws and load cell. The jaws must have flat surfaces and must be wide enough to fit the sample (e.g., at least 2.54 cm wide). Also, the jaws should provide adequate force to ensure that the sample does not slip during testing. The load cell is selected so that the tensile response from the sample tested is between 25% and 75% of the capacity of the load cell used.

3. Calibrate the tester according to the manufacturer's instructions.

4. Set the distance between the grips at 25 mm.

5. Place the sample in the flat surface of the jaws such that the longitudinal axis of the sample is substantially parallel to the gauge length direction. Mount the sample with minimal slack. Set the slack preload at 0.02 N/cm. This means that the data collection starts when the slack is removed with a force of 0.02 N/cm. Strain is calculated based on the adjusted gauge length (lᵢₙᵢ), which is the length of the sample in between the grips of the tensile tester at a force of 0.02 N/cm. This adjusted gauge length is taken as the initial sample length, and it corresponds to a strain of 0%. Percent strain at any point in the test is defined as the change in length divided by the adjusted gauge length times 100%.

6(a). First cycle loading: Pull the sample to a strain of 50% at a constant cross head speed of 254 mm/min.

6(b). First cycle unloading: Hold the sample at 50% strain for 30 seconds and then return the crosshead to its starting position (0% strain) at a constant cross head speed of 254 mm/min. Hold the sample in the unstrained state for 1 minute.

6(c). Set from second cycle loading: Pull the sample at a constant cross head speed of 254 mm/min, till it reaches a load of 0.05 N/25.4 mm (0.020 N/cm). Record the extended gauge length (l_{w}). Next, return the crosshead to its starting position (zero strain) at a constant cross head speed of 254 mm/min. Set is defined as the strain at a second cycle load of 0.05 N/25.4 mm (0.020 N/cm). Calculate % set as indicated below.

6(d). Second cycle unload: Next, return the crosshead to its starting position (zero strain) at a constant cross head speed of 254 mm/min.

Percent Set is defined as the percent strain at a second cycle load of 0.05 N/25.4 mm (0.020 N/cm). Calculate % set as indicated below.

A computer data system records the force exerted on the sample during the test as a function of applied strain. From the resulting data generated, the following quantities are reported (note that loads are reported as force divided by the width of the sample and do not take into account the thickness of the sample):

1. Loads at 25% strain and 50% strain (N/cm)

2. % set (Percent Strain measured at a second cycle load of 0.02N/cm);

3. % set=(lₑₓₜ−lᵢₙᵢ)/lᵢₙᵢ*100%.

Five repetitions are done on each sample and the average and standard deviation reported.

The Hysteresis Test can be suitably modified depending on the expected attributes and/or properties of the particular material sample to be measured. For example, the Test can be suitably modified where a sample of the length and width specified above are not available from the subject article.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross-referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended that the appended claims cover all such changes and modifications, and that nothing in the foregoing description or the figures, but rather, only the appended claims, limit the scope of the invention.

What is claimed is:

1. A package associating at least first and second sets of disposable wearable absorbent articles each having a backsheet, a topsheet, an absorbent core disposed between the backsheet and the topsheet, a front waist edge, a rear waist edge and a pair of side leg edges,
wherein the articles of first set have a first structure, and the articles of the second set have a second structure, and the first structure differs from the second structure in one or more linear dimensions by at least 10 percent.

2. A package associating at least first and second sets of disposable wearable absorbent articles each having a backsheet, a topsheet, an absorbent core disposed between the backsheet and the topsheet, a front waist edge, a rear waist edge and a pair of side leg edges,
wherein the articles of first set have a first structure, and the articles of the second set have a second structure, and the first structure differs from the second structure in one more respects selected from the group consisting of:
leg band width WLB, barrier cuff width WBC, leg band length LLC, presence and number of leg band elastic members, size of leg band elastic members, material(s) of which leg band elastic members are formed, leg band edge profile, backsheet film crotch width WBS, backsheet film leg opening edge profile, fastening member length LF, fastening member top edge location LLF, fastening member profile/shape, hip edge length LHE, side panel chassis edge length LSPC, side seam longitudinal bonding length, side panel longitudinal bonding length, overall length L, front rise FR, rear rise RR, front and/or rear waist edge profiles, longitudinal location of one or more of waist elastic members, presence and number of waist elastic members, size of waist elastic members, material(s) of which waist elastic members are formed, and rear waist region width WWR, and combinations thereof.

3. The package of claim 1 wherein the first structure differs from the second structure in one or more respects selected from the group consisting of leg band width WLB, barrier cuff width WBC, backsheet film crotch width WBS, fastening member length LF, fastening member top edge location LLF, hip edge length LHE, side panel chassis edge length LSPC, side seam longitudinal bonding length, side panel longitudinal bonding length and rear waist region width WWR, and combinations thereof, and for said at least one respect, the difference is at least 10%.

4. The package of claim 1 wherein the first structure differs from the second structure in leg band length LLC and the difference is at least 10%.

5. The package of claim 2 wherein the first structure differs from the second structure in one or more respects selected from the group consisting of overall length L, front rise FR, rear rise RR, and longitudinal location of one or more of waist elastic members, and combinations thereof, and for said at least one respect, the difference is at least 5%.

6. The package of claim 1 wherein the package associates a total quantity of disposable wearable absorbent articles; wherein the first set comprises a first number of first articles having the first structure, and the second set comprises a second number of articles having the second structure; wherein the total quantity of disposable wearable absorbent articles associated by the package is at least 18; and wherein a ratio of the first number of articles to the second number of articles is from 1:2 to 1:1.

7. The package of claim 2 wherein the package associates a total quantity of disposable wearable absorbent articles; wherein the first set comprises a first number of first articles having the first structure, and the second set comprises a second number of articles having the second structure; wherein the total quantity of disposable wearable absorbent articles associated by the package is at least 18; and wherein a ratio of the first number of articles to the second number of articles is from 1:2 to 1:1.

8. The package of claim 6 wherein the articles of the first set each bear a first differentiating indicium, and the articles of the second set each bear a second differentiating indicium visually distinguishable from the first differentiating indicium.

9. The package of claim 7 wherein the articles of the first set each bear a first differentiating indicium, and the articles of the second set each bear a second differentiating indicium visually distinguishable from the first differentiating indicium.

10. The package of claim 6 wherein the articles of the first set and the articles of the second set are intermingled.

11. The package of claim 7 wherein the articles of the first set and the articles of the second set are intermingled.

12. The package of claim 1 wherein each of the disposable wearable absorbent articles further comprises a pair of elasticized barrier cuffs.

13. A method of reducing incidence of marking, skin irritation and/or chafing of the skin of a wearer of disposable wearable absorbent articles, comprising the steps of:
providing at least first and second sets of disposable wearable absorbent articles each having a backsheet, a topsheet, an absorbent core disposed between the backsheet and the topsheet, a pair of elasticized barrier cuffs having barrier cuff edges, a front waist edge, a rear waist edge and a pair of side leg edges,
wherein the articles of the first set have a first structure, and the articles of the second set have a second structure, and the first structure differs from the second structure in one more respects selected from the group consisting of:
leg band width WLB, barrier cuff width WBC, leg band length LLC, presence and number of leg band elastic members, size of leg band elastic members, material(s) of which leg band elastic members are formed, leg band edge profile, backsheet film crotch width WBS, backsheet film leg opening edge profile, fastening member length LF, fastening member top edge location LLF, fastening member profile/shape, hip edge length LHE, side panel chassis edge length LSPC, side seam longitudinal bonding length, side panel longitudinal bonding length, overall length L, front rise FR, rear rise RR, front and/or rear waist edge profiles, longitudinal location of one or more of waist elastic members, presence and number of waist elastic members, size of waist elastic members, material(s) of which waist elastic members are formed, and rear waist region width WWR, and combinations thereof; and
applying an article of the first set to the wearer;
at the time for a change of articles on the wearer, replacing the article of the first set with an article of the second set.

14. A method for manufacturing a package associating at least first and second sets of disposable wearable absorbent articles each having a backsheet, a topsheet, an absorbent core disposed between the backsheet and the topsheet, a front waist edge, a rear waist edge and a pair of side leg edges, the package associating a total quantity of disposable wearable absorbent articles,
wherein the first set comprises a first number of first articles having a first structure, and the second set comprises a second number of articles having a second structure, and the first structure differs from the second structure and the first and second structures are manufactured sequentially on the same manufacturing line, comprising the steps of
providing a web material; and
conveying the web material in a machine direction into the nip of a die roller mechanism comprising a die roller, the die roller comprising one or more cutting blades;
wherein the one or more cutting blades has a first cutting profile adapted to cut the web material along a first cutout profile for a first article component, and a second cutting profile adapted to cut the web material along a second cutout profile for a second article component sequentially following the first article component as the web material moves in the machine direction.
